(19) United States
(12) Patent Application Publication
(10) Pub. No.: US 2004/0006315 A1
(43) Pub. Date: Jan. 8, 2004

(54) Syringe structure

(76) Inventor: Pi-Chang Lo, Taoyuan City (TW)

Correspondence Address: M.K. Meditech Co., Ltd., P.O. Box No.6-57, Chung-Ho, Taipei, 235, TW

(21) Appl. No.: 10/189,470

(22) Filed: Jul. 8, 2002

(51) Int. Cl. A61M 5/315

(52) U.S. Cl. 604/228; 604/110

(57) ABSTRACT

A syringe structure includes a tube plug body embedded at an axle center of a rubber head at the front end of a push rod structure inside a hollow needle barrel; furthermore, a retaining ring is disposed in a corresponding area at the front end inside the hollow needle barrel. When the push rod pushes forwardly to a terminal end, the retaining ring retains a retaining lug at the front end of the tube plug body. When the push rod pulls backward reversely, the tube plug body immediately detaches from the rubber head thereby forming an air groove to make the syringe loose an vacuum air-tightness and unable to extract the medicine. Furthermore, a safety retaining tab and a retaining slot are disposed at the distal end of the push rod to provide a safety device before application so as to eliminate syringe self-destruction caused by any accidental pressing.

### BACKGROUND OF THE INVENTION

1) Field of the Invention

The present invention relates to a syringe structure, more especially to a syringe structure designed to have a tube plug body disposed in a push rod structure and a rubber head as well as detachable after use to disable the syringe to be airtight thereby preventing the reuse of the syringe. In addition, a retaining slot and a safety retaining tab are disposed at a press end; for injecting, it is necessary to first press the safety retaining tab into the retaining slot for completely pushing the push rod thereby providing a safe design to eliminate syringe self-destruction caused by any accidental press on the push rod.

2) Description of the Prior Art

With more advanced medical technique, people nowadays emphasizes more on the issue of sanitation in applying medical and nursing apparatus. For years, many incidents of medical workers or patients getting infected directly or indirectly during a medical treating procedure occur unceasingly. Most of the indirect infection is caused by unclean medical apparatus due to incomplete sterilization or repetitive use of the supposedly discarded objects. For example, an injection syringe directly contacted with the patient is supposed to be discarded right after use and not to be reused for another patient. However, some medical units only throw away the needle head portions but reuse the needle barrel portions. That is very dangerous because although the needle barrel portions do not touch the patient directly, the blood probably refluxes during the injection procedure and relatively carries the bacteria or virus into the needle barrel. It is very possible that the next patient injected by the syringe with the same needle barrel would be contaminated through the bacteria or virus mixed inside the medicine. That not only deprives the patient's rights and interests, but also endangers his or her life.

### SUMMARY OF THE INVENTION

The primary objective of the present invention is to solve and prevent the existence of the abovementioned shortcomings. The present invention destructs an airtight space formed by a push rod and a rubber head disposed inside a syringe to necessarily and tightly affix unto the inner wall of the syringe for extracting and pushing the medicine so as to achieve the objective of disabling the syringe for repetitive use.

Another objective of the present invention is to completely eliminate syringe self-destruction caused by any accidental press on the push rod; therefore, a retaining slot and a safety retaining tab are disposed at a press end of the push rod for corresponding engagement; for injecting, the safety retaining tab must press into the retaining slot to push and the push rod completely for application.

In order to achieve the abovementioned objective, the present invention has a through hole disposed at the front end of the push rod and in the rubber head area; a tube plug body is plugged at the front end of the through hole for controlling the tight seal state of the rubber head; when the rubber head and the push rod structure produce an air groove due to the detachment of the tube plug body, the needle barrel and the push rod are unable to from an airtight state thereby become self-destructive.

To enable a further understanding of the structural features and the technical contents of the present invention, the brief description of the drawings below is followed by the detailed description of the preferred embodiment.

### BRIEF DESCRIPTION OF THE DRAWINGS

FIG. 1 is a pictorial and schematic drawing of external view of the present invention.

FIG. 2A is an exploded and schematic drawing of the structure of the present invention.

FIG. 2B is a planar and schematic drawing of the present invention.

FIG. 3A is the first schematic drawing of the application of the present invention.

FIG. 3B is the second schematic drawing of the application of the present invention.

FIG. 3C is the third schematic drawing of the application of the present invention.

FIG. 3D is the fourth schematic drawing of the application of present invention.

### DETAILED DESCRIPTION OF THE PREFERRED EMBODIMENTS

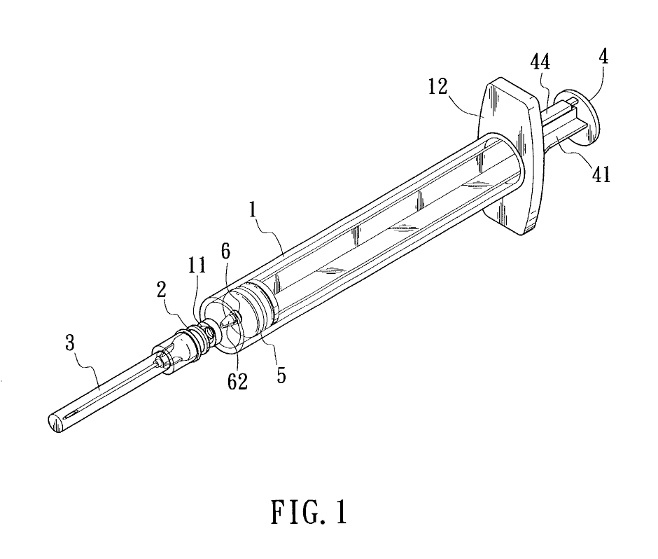

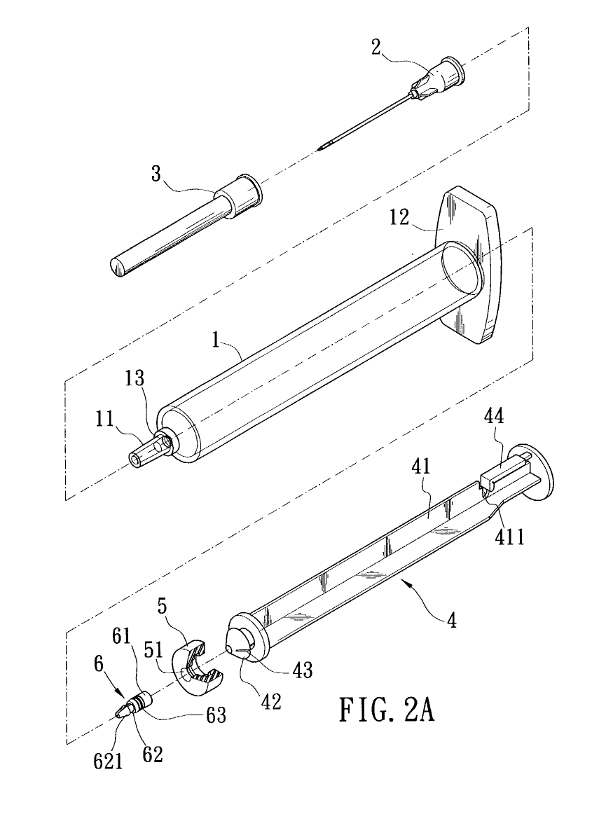

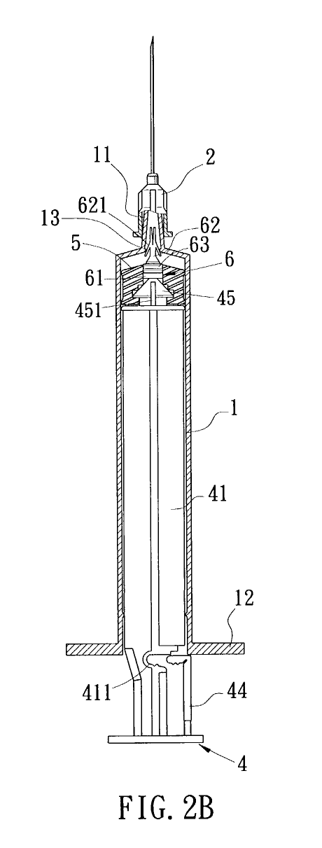

FIGS. 1, 2A and 2B show the external, pictorial, exploded, structural, planar and schematic drawings of the present invention; as indicated, the present invention mainly comprises a hollow needle barrel (1), a needle base (2), a needle case (3) and a push rod structure formed by a push rod (4) and a rubber head (5); wherein the front end of the hollow needle barrel (1) shrinks into a drop to form a adapter (11) with a drop retaining ring (13) disposed therein; the tail end of the needle barrel (1) forms a hold portion (12) for the user to hold; the needle base (2) is cased onto the adapter (11) for further casing the needle case (3); the function of the needle case (3) is to prevent any unexpected needle injury caused by the needle base (2).

The push rod (4) entering from the opening at the tail portion of the hollow needle barrel (1) is received and able to make an axial movement therein; a plurality of pieces (41) in radiant shapes are disposed on the push rod (4); furthermore, a retaining head (42) is formed at the front end of the push rod (4) and air escape grooves (43) are disposed on two opposite sides of the retaining head (42).

In addition, the rubber head (5) inserted onto the retaining head (42) at the front end of the push rod (4) tightly affixes unto the inner wall of the hollow needle barrel (1) allowing the interior portion of the hollow needle barrel (1) to form an airtight structure space. Furthermore, the axle center of the rubber head (5) is disposed with a through hole (51); a tube plug body (6) is embedded inside the through hole (51) to fixedly connect with an end portion of the retaining head (42) for sealing the through hole (51) thereby maintaining the required tight-sealing and leakage stopping normal condition of the rubber head (5).

Furthermore, the tube plug body (6) mainly comprises a leakage stop portion (61) disposed with a plurality of tight seal rings (63); retaining lugs (62) are disposed on the tight seal rings (63); wherein the leakage stop portion (61) fixedly connects with the retaining head (42) of the push rod (4) and tightly affixes with the through hole (51) so as to meet the requirement of sealing the through hole (51) in a normal condition.

However, the push portion (the distal end) of the push rod (4) is disposed with a safety retaining tab (44) and a corresponding retaining slot (411) to provide a safe device; wherein the function of the safety retaining tab (44) is to prevent any accidental press on the push rod (4) which might lead to a self-destruction caused by an error retaining between the retaining lug (62) and the drop retaining ring (13).

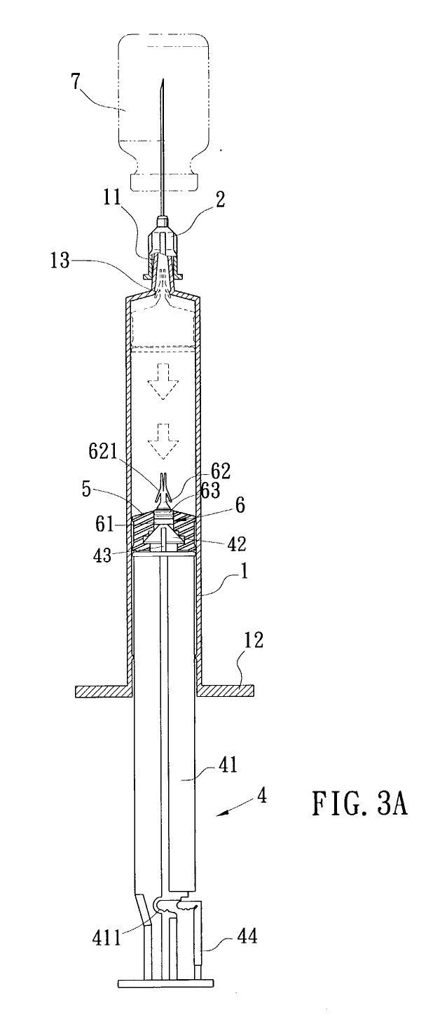

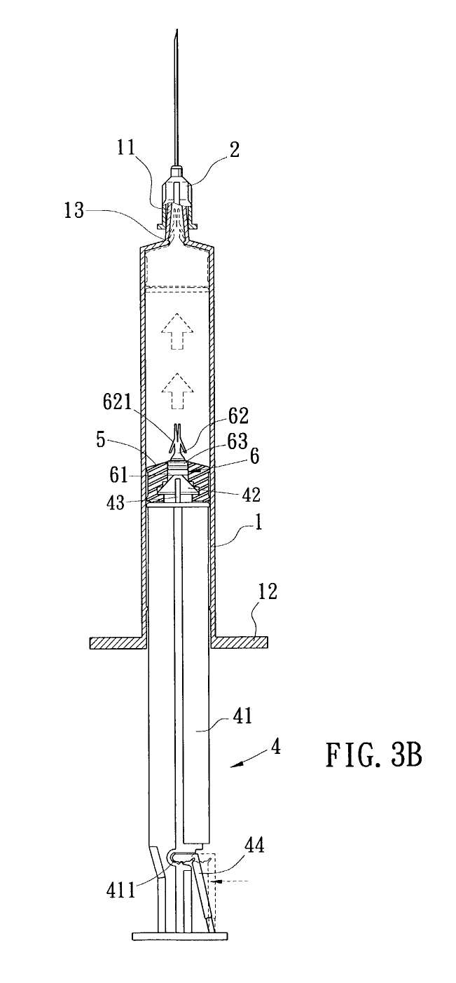

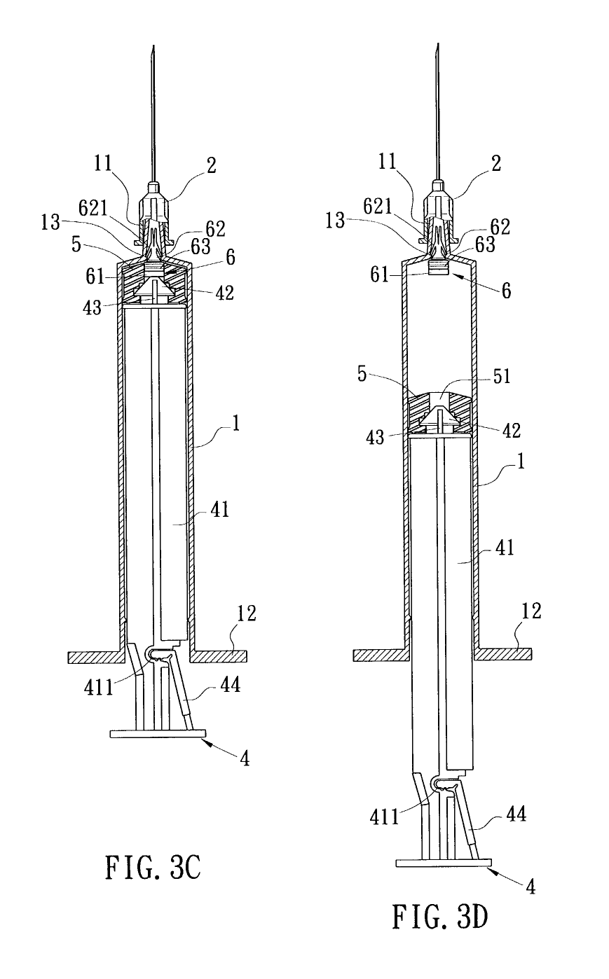

FIGS. 3A, 3B, 3C and 3D show the first, second and third schematic drawings of the application of the present invention. As indicated, when a medical worker uses the present invention, the involved application steps are exactly the same as that of using a traditional injection syringe. First, the needle base (2) inserts into a medicine bottle (7) filled with medicine to be injected; since the rubber head (5) of the push rod (4) tightly affixes unto the inner wall of the hollow needle barrel (1), pulling the push rod structure from the bottom portion of the hollow needle barrel (1) sucks the medicine inside the medicine bottle (7) into the hollow needle barrel (1) through the vacuum state of the airtight space for injection.

After a slight adjustment, the medical worker is ready to inject the medicine into the patient's body. As the same, under the situation that the rubber head (5) and the hollow needle barrel (1) affix tightly, it is necessary to first press the safety retaining tab (44) into the retaining slot (411) for pushing the push rod (4) completely.

When the medical worker pushes the push rod (4), the medicine injects into the patient's body via the needle base (2) and through the rubber head (5). The injection is completed when the rubber head (5) pushes against the bottom portion of the hollow needle barrel (1). Then the medical worker withdraws the needle base (2) from the patient's body. At this moment, the rubber head (5) pushes against the bottom portion of the hollow needle barrel (1) and the retaining lug (62) of the tube plug body (6) easily enters into the adapter (11) through a slanted guide side (621) thereon. The reverse retaining portion on the retaining lug (62) retains correspondingly on the drop retaining ring (13) to make the tube plug body (6) clip in the adapter (11) thereby forming a retaining state.

At the mean time, the medical worker once again withdraws the push rod (40). The retaining lug (62) thereof is retained in the area of the drop retaining ring (13) of the adapter (11) in an inserted and positioned state. Therefore, pulling the push rod (4) detaches the retaining lug (62) from the through hole (51) of the rubber head (5) allows the through hole (51) to return to a communicated state and connect with the air escape groove (43) thereby creating an air channel in the structure of the rubber head (5) and the push rod (4) due to the detachment of the tube plug body (6) so as to disable the hollow needle barrel (1) and the push rod (4) structure to form air-tightness. Therefore, if anyone tries to reuse the present invention by pulling or pushing the push rod (4), the air exits continuously via the air escape groove (43) of the retaining head (42) and through the through hole (51); no airtight space can then be formed between the rubber head (5) and the hollow needle barrel (1). Thus, the user is unable to reuse the present invention for sucking or pushing the medicine because it is necessary to have an airtight structure space for implementing the extraction of the syringe and the medicine injection thereby further preventing the repetitive use of the syringe and eliminating the possible occurrence of contamination.

It is of course to be understood that the embodiment described herein is merely illustrative of the principles of the invention and that a wide variety of modifications thereto may be effected by persons skilled in the art without departing from the spirit and scope of the invention as set forth in the following claims.

1. A syringe structure mainly comprising:
a hollow needle barrel with a adapter disposed in the front end area thereof and a hold portion formed at the tail end thereof;
a needle base inserted on the adapter;
a case cover cased on the needle barrel;
a push rod and a rubber head inserted at the front end thereof and disposed in the hollow needle barrel to form a push rod structure; wherein a plurality of pieces in radiant shapes are disposed on the push rod; the rubber head tightly affixes with the inner wall of the needle barrel; it is characterized that a tube plug body is embedded in the axle center area to connect with a retaining head; a drop retaining ring is disposed inside the adapter of the needle barrel such that when the push rod is pushed forwardly, a retaining lug at the front end of the tube plug body corresponds to retain; when the push rod is pulled backwardly, the tube plug body breaks from the retaining head and detaches from the rubber head to make the push rod structure create an air groove due to the detachment of the tube plug body thereby disabling the needle barrel and the push rod structure to be airtight and become self-destructive.

2. The syringe structure according to claim 1, wherein air escape grooves are correspondingly disposed on two sides of the retaining head at the front end of the push rod to communicate with a through hole at the axle center of the rubber head after the tube plug body and the retaining head break and separate from each other.

3. The syringe structure according to claim 1, wherein the tube plug body mainly comprises a leakage stop portion disposed with tight seal rings and retaining lugs.

4. The syringe structure according to claim 3, wherein, one end of the leakage stop portion is disposed with a plurality of tight seal rings and the other end fixedly connects with the retaining head.

5. The syringe structure according to claim 3, wherein a slanted guide side is disposed on the retaining lug.

6. The syringe structure according to claim 1, wherein a safety retaining tab and a corresponding retaining slot are disposed at the press end of the push rod; the function of the safety retaining tab is to eliminate the self-destruction of the syringe caused by any accidental press on the push rod; when in use, it is necessary to first press the safety retaining tab toward the retaining slot to completely push the push rod forwardly thereby thoroughly retain the retaining lugs and the retaining rings of the tube plug body to form self-destruction.
